(19) United States
(12) Patent Application Publication
(10) Pub. No.: US 2014/0163102 A1
(43) Pub. Date: Jun. 12, 2014

(54) MEDICAL DRESSING FOR RESPIRATORY EPITHELIAL CELLS

(71) Applicant: National Taiwan University, Taipei (TW)

(72) Inventors: TAI-HORNG YOUNG, Taipei City (TW); TSUNG-WEI HUANG, Taipei City (TW); HSU-HSIEN CHANG, Taipei City (TW)

(73) Assignee: National Taiwan University, Taipei (TW)

(21) Appl. No.: 14/099,395

(22) Filed: Dec. 6, 2013

(30) Foreign Application Priority Data
Dec. 6, 2012 (TW) 101145941

(51) Int. Cl. A61K 31/203

(52) U.S. Cl. CPC A61K 31/203
USPC 514/559

(57) ABSTRACT

The present invention provides a medical dressing for respiratory epithelial cells comprising a biocompatible polymer and retinoic acid, wherein, based on total volume of the dressing, the polymer is 99% or more, and retinoic acid is 1% or less.

### CROSS-REFERENCE TO RELATED APPLICATION

This application claims priority to Taiwan Application No. 101145941, filed Dec. 6, 2012.

### BACKGROUND OF THE INVENTION

1. Field of the Invention

The present invention relates to a medical dressing, and more particularly to a medical dressing for a respiratory tract.

2. Description of the Related Art

Comprising mucociliary epithelium, respiratory epithelium serves as an important defense mechanism against inhaled toxins, pathogens, and particles. Discontinuity of the epithelium, e.g., CSF rhinorrhea, may cause headache and central nervous system infection. Septal perforation may cause inflammation, nasal crusting, bleeding, and whistling while breathing. To date, various surgical repairs for the septal perforation are proposed, such as advancement mucosal flaps, lateral nasal wall flaps, and autografts using temporal fascia. However, several drawbacks are encountered, i.e., donor site morbidity, tissue shortage, and retention of the original characteristics of the donor tissue.

Recently, several biocompatible biomaterials are developed to serve as grafts. Most graft materials are composed mainly of collagen, however, several disadvantages of collagen, e.g., fast biodegrading rate, low mechanical strength, and extremely high cost, restrict its clinical usage.

Therefore, there is still a need for a biomedical material applicable for respiratory tract.

### SUMMARY

The present invention provides a medical dressing of respiratory tract comprising a hiocompatible polymer and a retinoic acid, wherein, based on total volume of the medical dressing, the polymer is 99% or more, and retinoic acid is 1% or less.

The present invention also provides a method for regulating growth of respiratory epithelial cells comprising administering a mixture comprising a biocompatible polymer and a retinoic acid, wherein, based on total volume of the mixture, the polymer is 99% or more and retinoic acid is 1% or less, and contacting the mixture and the respiratory epithelial cells for a predicted time period.

### BRIEF DESCRIPTION OF THE DRAWINGS

FIG. 1A illustrates the residual amount of retinoic acid in the RA-HAm mixture.

FIG. 1B illustrates the amount of retinoic acid in the medium.

FIG. 2 illustrates the morphology of RECs at confluence on (A) HAm and (B) RA-HAm.

FIG. 3 illustrates the result of cell activity examination.

FIG. 4 illustrates the scanning electron micrographs of RECs on (A) HAm and (B) RA-HAm.

FIG. 5 illustrates the expression of MUC5AC.

FIG. 6 illustrates the expression of AQPS.

### DETAILED DESCRIPTION OF THE EMBODIMENTS

In the medical dressing of the present invention, the polymer, based on total volume of the medical dressing, is 99% or above but less than 100%, for example, 99.1%, 99.3%, 99.5%, 99.7%, 99.9%, 99.99% or a value between any two points of the above values. The medical dressing of the present invention also contains 1% or less to of the retinoic acid based on total volume of the medical dressing, such as 0.9%, 0.7%, 0.5%, 0.3%, 0.1%, 0.05%, 0.01% or a value between any two points of the above values.

The medical dressing has an effect of sustained release of the retinoic acid. In one embodiment, the medical dress is able to release the retinoic acid with a stable rate during a long time period. In one embodiment, the sustained release is conducted for several days, such as 7 days to 10 days.

The medical dressing of the present invention can be solid, solution, and/or gel. Optionally, a carrier can be applied to the medical dressing. The medical dressing can be applied in the respiratory tract, especially for the respiratory epithelial cells. For example, the medical dressing can he used as a wound care dressing, an adhesive patch, a filler material, a transplant and the like. In one embodiment of the wound healing of the respiratory tract, the medical dressing is directly applied to cover the wound, in which the polymer blocks the contact between the wound and the environment and provides a closed condition with moisture, and the retinoic acid is released to the wound with a sustained and stable rate, so that the repair, development and differentiation of the respiratory epithelial cells can be induced to facilitate the wound healing.

Accordingly, the present invention also provides a method for regulating growth of respiratory epithelial cells comprising administering a mixture comprising a biocompatible polymer and a retinoic acid, wherein, based on total volume of the mixture, the polymer is 99% or more and retinoic acid is 1% or less, and contacting the mixture and the respiratory epithelial cells for a predicted time period, so that the adhering, development and differentiation of respiratory epithelial cells can be induced.

The respiratory epithelial cells treated by the mixture or the dressing o the present invention are able to differentiate to cilia.

In the present method, the mixture comprises 99% or more of a polymer and 1% or less of retinoic acid, based on total volume of the mixture. In a preferred embodiment, the polymer is 99.9% or above and retinoic acid is 0.1% or less.

In one embodiment, the respiratory epithelial cells contact with the mixture for a predicted time period. The contacting time is long enough to release a sufficient amount of retinoic acid from the mixture to the cells. In one embodiment. the contacting time is 4 hours or above. such as 8 hours, 16 hours, 1 day, 3 days, 7 days, 10 days and the like.

In one embodiment, the polymer is selected from hyaluronic acid, chitosan, collagen, or any combination thereof. In one embodiment, the hyaluronic acid, the chitosan and the collagen can be modified, or can be the derivatives.

Hyaluronic acid is a polysaccharide formed by repeated units composed of disaccharides, i.e. D-glucuronic acid and N-acetyl glucosamine. In one embodiment the hyaluronic acid has a molecular weight of about 5 KDa to about 20,000 KDa.

In another embodiment, the hyaluronic acid may be a hyaluronic acid ester such as hyaluronic acid ethyl ester, hyaluronic acid propyl ester, hyaluronic acid aromatic ester (e.g. hyaluronic acid phenyl ester), hyaluronic acid acrylic ester, hyaluronic acid carboxylic ester and the like; crosslinked hyaluronic acids such as hyaluronic acids treated by a crosslinking agent including divinylsulfone (DVS), 1-ethyl-3-(3-dimethyl aminopropyl)carbodiimide (EDC), formaldehyde, epoxides, metal ions and the like; modified or branched hyaluronic acids including hyaluronic acid modified or branched by such as glycidyl methacrylate, polylactide-poly(ethylene glycol) copolymer and the like. The above component can be used alone or in combination. In a preferred embodiment, the hyaluronic acid ester used.

Chitosan is a polysaccharide mainly composed of glucosamine. In one embodiment, chitosan may be chitosan alkyl derivatives including such as ethyl, butyl, octyl, hexadecyl, hydroxyethyl derivatives and the like; chitosan monosaccharide derivatives such as chitosan covalent bound with glucose, fructose, semigalactose, glucosamine and the like; chitosan disaccharide derivatives chitosan such as chitosan covalent bound with lactose, maltose, cellobiose and the like. The above component can be used alone or in combination.

In embodiments, the type of collagen is not limited, which can be type I, type II, type III, type IV and the like. In embodiments, the collagen can be such as fiber, tube, porous type, film, sponge, injection formulation and the like.

In the present invention, the release of retinoic acid from the dressing is as a function of time and is not affected by the initial concentration of retinoic acid. Accordingly, the dressing of the present invention is able to provide dynamic regulation of the release of retinoic acid.

Examples of the medical dressing of the present invention are further described hereafter.

### EXAMPLE

### 1. Preparation of a Mixture of Retinoic Acid and Hyaluronic Acid Derivative (RA-HAm)

All steps were conducted in dark. 180 mg of benzyl esters of hyaluronic acid (HYAFF)(Fidia Advanced Biomaterials, Italy) seas dissolved in 1 ml of dimethyl sulfoxide (DMSO) at room temperature. 10 μl of retinoic acid (10-3 M in ethanol) was added into the HYAFF solution and mixed homogeneously. A thin layer of the above mixture (150 μl/cm2) was then spread on a glass plate. Next, ethanol was added in the proportion of 100 folds of the volume of the mixture to precipitate HYAFF. The polymer sheet was removed gently, and dried under vacuum and dark at room temperature for 12 hours. Before use for culturing, these RA-HAm membranes were rinsed with phosphate buffered saline (PBS).

### 2. Detection of the Release of RA

The prepared RA-HAm membrane was put on the Transwell membrane insert, and the medium was changed daily. The RA-HAm was dissolved in 500 μl of DMSO, and absorption of wavelength 380 nm was then detected by using an HASA meter to obtain the residual amount of retinoic acid in the RA-HAm membrane. The amount of retinoic acid in the collected medium was also detected. All steps were conducted in dark.

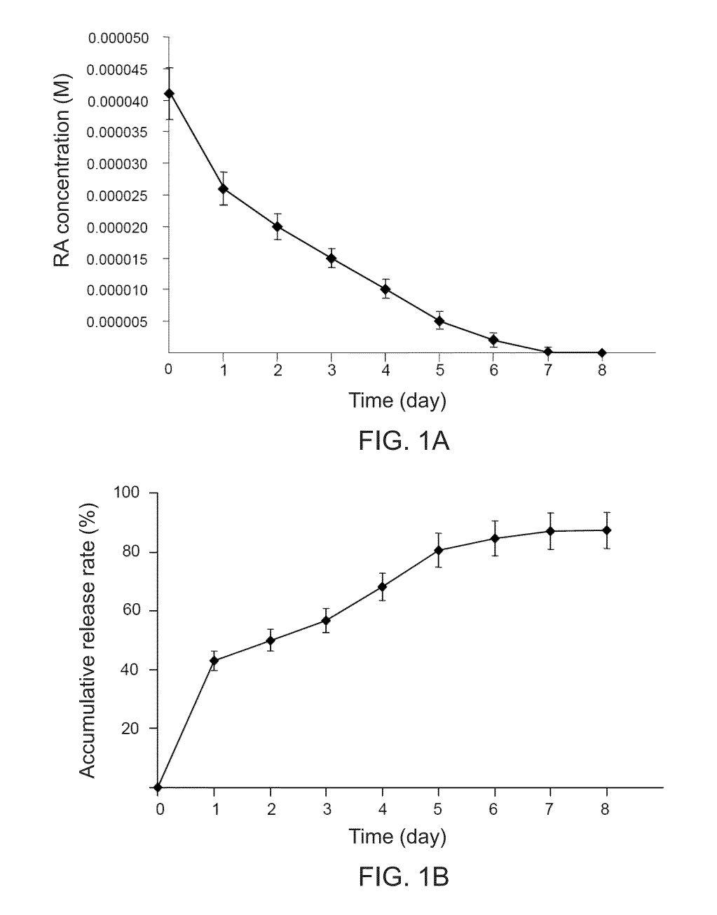

FIG. 1A illustrates the residual amount of retinoic acid in the RA-HAm. The initial concentration was 420 μM. A slightly higher amount of RA was released in the first day, and then a slow and continuous release of RA for about 7 days was observed. FIG. 1B illustrates the accumulative amount of retinoic acid in the medium. About 87% of RA was accumulated in the medium in the 7th day, It showed that the RA-HAm membrane was able to release RA stably and continuously.

### 3. Isolation and Culture of Human Respiratory Epithelial Cells

Isolation and culture of human respiratory epithelial cells were referring to Huang T W et al., Acta. Biomater (2010), 6: 1191-9 and Huang T W et al., Laryngoscope (2009), 119: 2066-70. Human nasal inferior turbinates was obtained from patients undergoing septomeatoplasty. Tissues were treated with 0.5% Pronase (type XIV protease, Sigma-Aldrich, LISA) in a 1:1 mixture of Dulbecco's modified Eagle's medium and Ham's nutrient F12 (DMEM/F12) supplemented with antibiotics (penicillin/streptomycin) for 16-20 hours at 4° C. The cell suspension eras filtered to remove cell aggregates and debris. After centrifugation, the cells were then suspended in DMEM/F12 bronchial epithelial growth medium (BEGM, Clonetics Corp., CA) (1:1) supplemented with antibiotics. Cells were pre-plated on a plastic dish at 37° C. for 1 hour to eliminate fibroblasts by differential attachment to plastic. The cells in suspension were then collected and resuspended in a culture medium at a concentration of 105 cells/ml. Next, 1.5 ml cell suspension was seeded on the RA-HAm membrane coated Transwell membrane insert with 2.6 ml of the medium deposited on the basolateral side. Cultures were maintained at 37° C. in a atmosphere of 5% carbon dioxide in air. Cells were grown submerged before confluence and the culture medium was changed after 48 hours first and every other day thereafter. Following confluence, ALI was created by removing the spucal medium and feeding the cultures only from the basolateral compartment.

### 4. Detection of Cell

### (1) Morphological Examination

Living, untreated cultures were observed under a light microscope. Furthermore, cultures were rinsed in PBS and in 0.1 M cacodylate buffer, fixed for 1 hour in 2.5% glutaraldehyde in 0.05 M cacodylate buffer, rinsed for 1 hour in 0.1 M cacodylate buffer, and then post fixed in 1% OsO4 in 0.05 M cacodylate buffer for 1 hour. Next, the specimens were dehydrated in a graded ethanol series and dried with the critical point technique with liquid CO2 and then sputter coated with gold to a nominal thickness of 25 mn. Finally, the samples were examined under a scanning electron microscope (SEM).

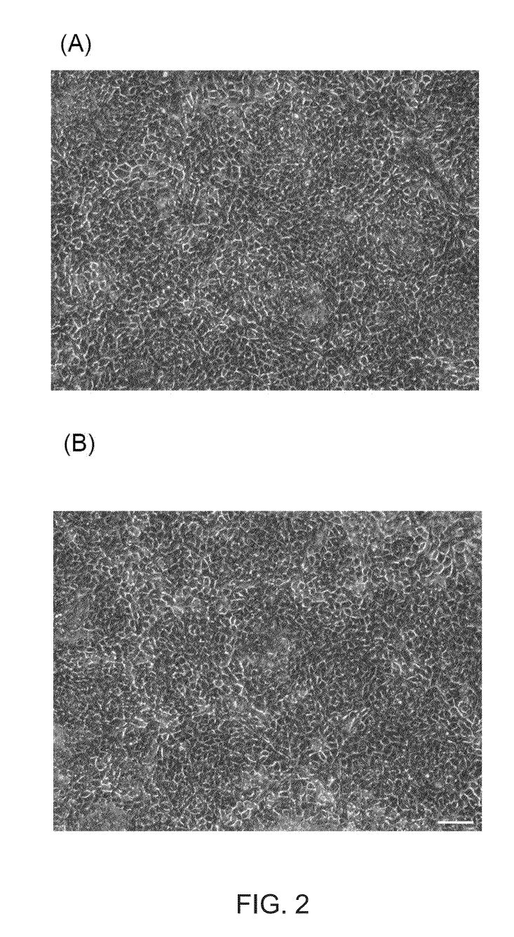

FIG. 2 illustrates the morphology of RECs at confluence on (A) HAm and (B) RA-HAm. Both groups showed epithelial-like morphology.

### (2) Cell Activity Examination

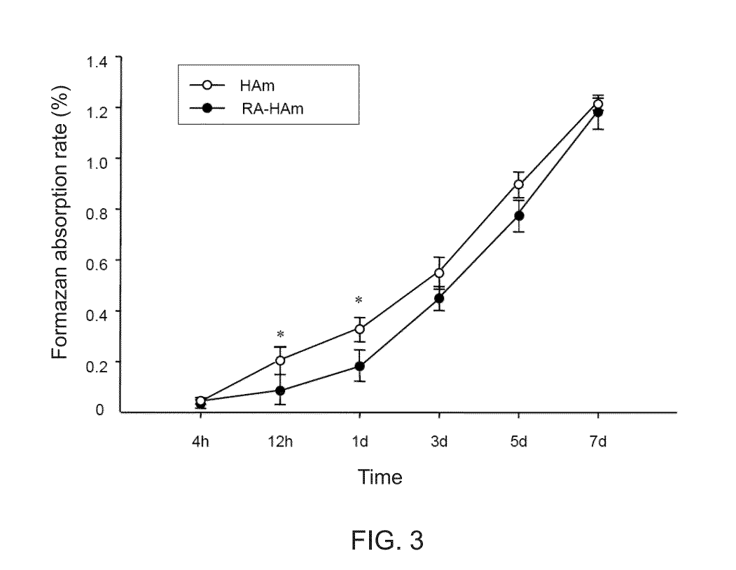

Cell activity at various time points was detected by MTT method, and the result was shown in FIG. 3. Absorption of formazan directly represented the cell numbers. Both of the RA-HAm and HAm groups showed that the number of cells increased continuously. The RA-HAm group did not show a rapid increase after the first day, but two groups showed similar proliferation rates after the third day and required 7-8 days to achieve confluence.

### (3) Cell Differentiation Examination

Following confluence, ALI was created as described above.

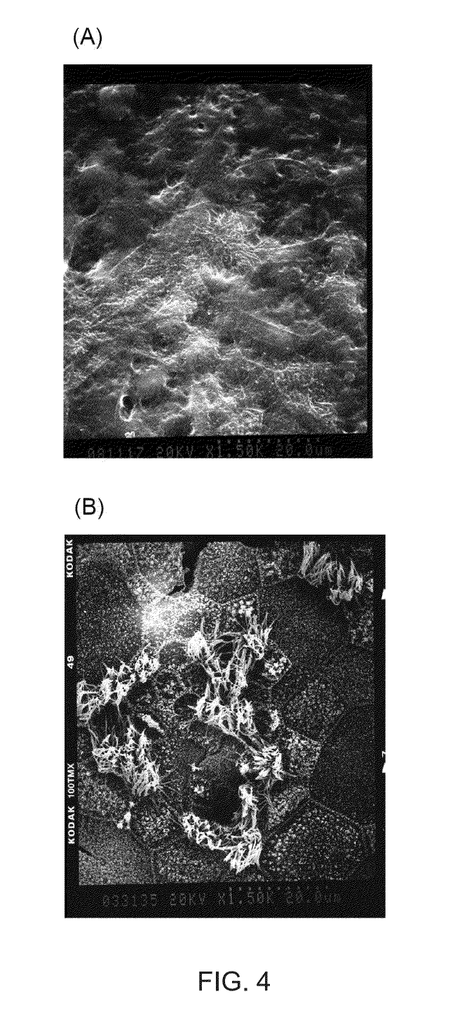

Cell morphology of RA-HAm and HAm groups observed under SEM was shown in FIG. 4, in which significant differences could be observed. In the RA-HAm group (FIG. 4(B)), RECs on ALI day 21 had numerous mature cilia and microvillus; in contrast, the HAm group (FIG. 4(A)) merely showed clustered ciliated cells.

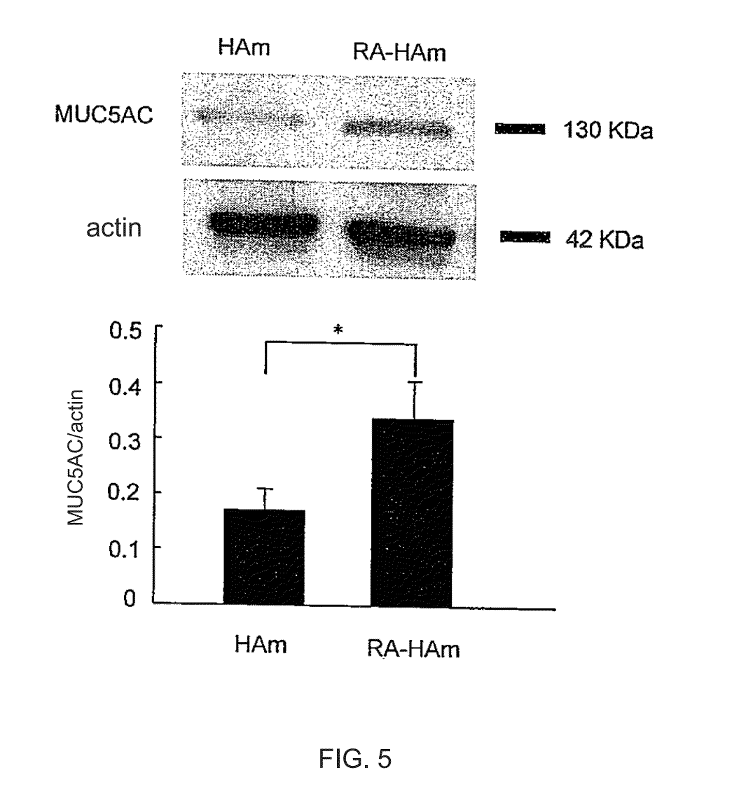

Protein expression of MUC5AC, the mucin of respiratory tract, was used to evaluate the differentiation of mucous membrane. The human respiratory epithelial cells on ALI day 21 were collected for detection of MUC5AC expression by using western blot; the result was shown in FIG. 5. MUC5AC expression of the RA-HAm group was higher than the HAm group. Actin was used as a control for quantification, The MUC5AC expression index of the HAm group and RA-HAm group were 0.17±0.04 and 0.34±0.07 respectively. It was significantly different between the two groups in the cell differentiation.

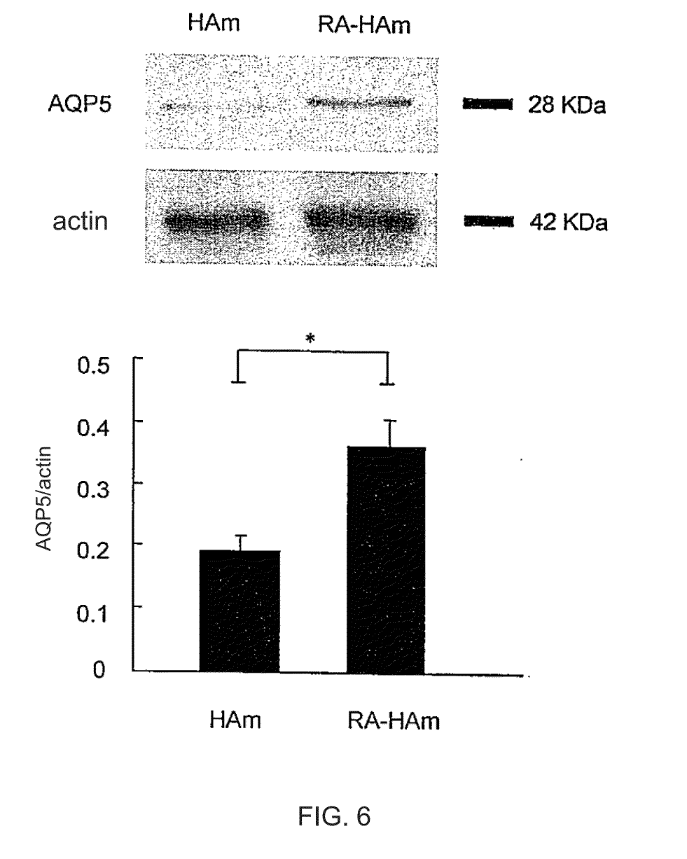

Furthermore, maintenance of water balance and periciliary fluid was important for the system function of mucous membrane and cilia. Protein expression of AQP5 was used to evaluate the above system function in a respiratory tract. The human respiratory epithelial cells on ALI day 21 were collected for detection of AQP5 expression by using western blot; the result was shown in FIG. 6. AQP5 expression of the RA-HAm group was higher than the HAm group. Actin was used as a control for quantification. AQP5 expression index of the HAm group and RA-HAm group were 0.2±0.02 and 0.38±0.04, respectively. It was significantly different between the two groups.

According to the above results, the mixture of a biocompatible polymer and a retinoic acid was able to continuously release retinoic acid to environment and facilitate proliferation of human respiratory epithelial cells as well as differentiation into cilia. Also, the mixture was able to assist and maintain the function of the nasal system of ucous membrane and cilia. The mixture of the present invention is a suitable medical dressing for a respiratory tract.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A medical dressing of respiratory tract comprising a biocompatible polymer and a retinoic acid, wherein, based on total volume of the medical dressing, the polymer is 99% or more, and retinoic acid is 1% or less, wherein the retinoic acid is sustain-released from the medical dressing.

2. The medical dressing of claim 1 wherein the polymer is selected from one of the group consisting of hyaluronic acid, chitosan and collagen.

3. The medical dressing of claim 1 wherein the hyaluronic acid is selected from one of the group consisting of hyaluronic acid, hyaluronic acid esters, crosslinked hyaluronic acids, modified hyaluronic acids, and branched hyaluronic acid.

4. The medical dressing of claim 3, wherein the hyaluronic acid ester comprises hyaluronic acid ethyl ester, hyaluronic acid propyl ester, hyaluronic acid aromatic ester, hyaluronic acid acrylic ester, hyaluronic acid carboxylic ester, or any combination thereof.

5. The medical dressing of claim 1, rerein the chitosan is selected from one of the group consisting of chitosan, chitosan alkyl derivatives, chitosan monosaccharide derivatives, and chitosan disaccharide derivatives.

6. The medical dressing of claim 1, wherein the retinoic acid has a concentration of 1 M to 1×10−10 M.

7. The medical dressing of claim 1, wherein the retinoic acid has a concentration of 1×10−3 M to 1×10−7 M.

8. The medical dressing of claim 1, wherein the retinoic acid has a concentration of 6×10−5 M to 1×10−6 M.

9. A method for regulating growth of respiratory epithelial cells comprising administering a mixture comprising a biocompatible polymer and a retinoic acid, wherein based on total volume of the mixture, the polymer is 99% or more and retinoic acid is 1% or less, and contacting the mixture and the respiratory epithelial cells for a predicted time period.

10. The method of claim 9, therein the retinoic acid is sustain-released from the mixture.

11. The method of claim 9, wherein the respiratory epithelial cells adhere to the mixture, and then develop and differentiate.

12. The method of claim 9, wherein the polymer is selected from one of the group consisting of hyaluronic acid, chitosan, and collagen.

13. The method of claim 9, wherein the retinoic acid has a concentration of 1 M to 1×10−10 M.
